(19) United States
(12) Patent Application Publication
(10) Pub. No.: US 2009/0011978 A1
(43) Pub. Date: Jan. 8, 2009

(54) Use of Surfactant Preparations for the Treatment of Surgical Adhesions

(75) Inventors: Dietrich Hafner, Konstanz (DE); Ralf Harand, Reichenau (DE); Eva Ammon, Allensbach (DE); Ernst Sturm, Konstanz (DE); Klaus Eistetter, Konstanz (DE); Friedemann Taut, Konstanz (DE); Thomas Muller, Konstanz (DE)

Correspondence Address: NATH & ASSOCIATES PLLC, 112 South West Street, Alexandria, VA, 22314, US

(73) Assignee: Atlanta Pharma AG, Konstanz (DE)

(21) Appl. No.: 10/568,816

(22) PCT Filed: Aug. 27, 2004
(86) PCT No.: PCT/EP2004/051947

§ 371 Date: Feb. 21, 2006

(30) Foreign Application Priority Data
Aug. 28, 2003 (EP) 03019446.8

(51) Int. Cl. A61K 38/17; A61P 17/02

(52) U.S. Cl. 514/12

(57) ABSTRACT

The invention describes the use of surfactant comprising phospholipids and pulmonary surfactant proteins for the treatment of surgical adhesions.

### TECHNICAL FIELD OF THE INVENTION

The invention relates to the novel use of surfactant preparations for the treatment of surgical adhesions.

### PRIOR ART

It is generally accepted that the function of pulmonary surfactants is to lower the surface tension at the air to water interface of the alveoli. For many years, it has proven suitable to treat IRDS (Infant Respiratory Distress Syndrome) by introducing pulmonary surfactant preparations into the lungs of premature babies. It is also known from pilot studies that pulmonary surfactant preparations are clinically effective in ALI (Acute Lung Injuries) including ARDS (Adult Respiratory Distress Syndrome) [survey, for example, B. Lachmann, D. Gommers and E. P. Eijking: Exogenous surfactant therapy in adults, Atemw.-Lungenkrkh. 1993, 19: 581-91; D. Walmrath et al.: Bronchoscopic surfactant administration in patients with severe adult respiratory distress syndrome and sepsis, Am. J. Respir. Crit. Care Med. 1996, 154: 57-62; T. J. Gregory et al.: Bovine surfactant therapy for patients with acute respiratory distress syndrome, Am. J. Respir. Crit. Care Med. 1997, 155: 1309-15].

It is also known from prior art that phospholipid preparations may be used for the prevention of surgical adhesions. WO 91/12026 discloses a method of reducing or preventing of unwanted surgical adhesions by means of coating tissue with a phospholipid, such as phosphogylcerides, phosphoglycolipids, phosphodiol lipids or phosphosphingolipids, preferably a phosphatidylcholin as lecithin, in suspension or solution in a surgically acceptable carrier, such as for example, water, saline, or propylene glycol, or mixture thereof.

WO 99/51244 describes the use of surface active phospholipids in reducing the probability of adhesions following surgery. Preferably, it refers to powdered formulations comprising phospholipids [e.g. DPPC (dipalmitoylphosphatidylcholine), unsaturated PG (phosphatidylglycerol) alone or at various ratios thereof] to prevent post-surgical adhesions.

U.S. Pat. No. 6,133,249 describes a method of lubricating mammalian joints using a liquid composition comprising phospholipids dispersed in propylene glycol.

WO 03/000344 discloses the use of liquid, semi-liquid or pasty compositions of certain phospholipids, such as DPPC, DPPC and PG, or DPPG, dispersed in a physiologically acceptable carrier for reducing the risk of surgical adhesions.

### SUMMARY OF THE INVENTION

Present invention refers to the use of a further pharmaceutical preparation for the prophylaxis of surgical adhesions or for prevention of the probability of surgical adhesions in patients in need thereof.

Surprisingly it has been found that phospholipid preparations additionally comprising surfactant proteins are equal to or better than known phospholipid preparations in the treatment of surgical adhesions. It has also surprisingly been found that powdered surfactant preparations fit particularly for the treatment of surgical adhesions.

In a first embodiment of present invention, there is provided the use of a surfactant preparation comprising at least one phospholipid, pulmonary surfactant proteins SP-B and/or SP-C and/or modified derivatives thereof and optionally excipients for the production of a medicament for the treatment of surgical adhesions. In particular, the use of surfactant preparations is preferred wherein the pulmonary surfactant protein is a recombinantly prepared pulmonary surfactant protein. The use of a modified derivative of a pulmonary surfactant protein is preferred and rSP-C (FF/I) is particularly preferred in such surfactant preparations.

In a further embodiment of present invention there is provided the use of a powdered surfactant preparation comprising at least one phospholipid, pulmonary surfactant proteins SP-B and/or SP-C and/or modified derivatives thereof and optionally excipients for the production of a medicament for the treatment of surgical adhesions. Particularly, powdered surfactant preparations obtainable by spray-drying are preferred.

In a further embodiment of present invention, there is provided a method for treating surgical adhesions in a patient in need thereof, the method comprises the step of administering the surfactant preparation comprising at least one phospholipid, pulmonary surfactant protein and optionally excipients to the patient in need thereof. Particularly preferred is such a method for treating surgical adhesions in a patient in need thereof, whereby the surfactant preparation is administered topically.

In a further embodiment of present invention there is provided a pharmaceutical composition comprising a surfactant preparation suited for the treatment of surgical adhesions, wherein the surfactant preparation comprises at least one phospholipid, pulmonary surfactant proteins SP-B and/or SP-C and/or modified derivatives thereof and optionally excipients.

Present invention also refers to a commercial product comprising a customary secondary packaging, a primary packaging comprising a surfactant preparation of at least one phospholipid and pulmonary surfactant proteins SP-B and/or SP-C and, optionally, a package insert, the surfactant preparation being suitable for treating surgical adhesions in patients in need thereof.

### DETAILED DESCRIPTION OF THE INVENTION

The novel use of a pharmaceutical preparation, which is the subject of present invention, comprises the administration of a surfactant preparation comprising phospholipid and pulmonary surfactant protein to a patient in need thereof. The invention thus relates to the use of a surfactant preparation for the production of a medicament for the treatment of adhesions.

The term “adhesion” refers to surgical adhesions as well as to adhesions occurring without surgery. Surgery of the abdomen or thorax and other forms of skin injury (e.g. after trauma) and other wounds where adhesion of tissue should be prevented after suture involve the incision in the skin followed possibly by further incisions into deeper tissue. Upon completion of the surgery or after skin injury, the two edges of each incision are held together by sutures or other technical means to promote the healing process by enabling cells to proliferate and fuse together at the open ends. A problem arises when tissue adhesion does not only occur between the edges of the same tissue as produced by the incision, but also occurs between edges of adjacent, different tissues. These fibrous adhesions can vascularise to form so called tissue “bridges”, also known as “surgical adhesions”, which are tightly bound to each other and which represent adhesions of two tissues which normally slide over each other. They are most undesirable where they inhibit the relative movement of adjacent tissue surfaces and are often manifested as stiffness, or immobility. If motion is forced, surgical adhesions can result in pain or they may rupture to produce haemorrhage.

Present invention takes into consideration that tissue bridges do not form if there is no direct contact between adjacent tissues (e.g. in between the pleural cavity) and if adjacent tissue can move freely without friction. Therefore, an object of present invention is to deliver a surfactant preparation comprising at least one phospholipid, pulmonary surfactant proteins SP-B and/or SP-C and/or modified derivatives thereof and optionally excipients which is useful as sliding material and which allows tissue surfaces or tissues to move without friction.

Therefore, according to this invention, the term “treating” or “treatment” of surgical adhesions refers to the prophylaxis of surgical adhesions and/or to the prevention of the probability of surgical adhesions. Thus, present invention refers to the use of a surfactant preparation for the production of a medicament for the prophylaxis of surgical adhesions and/or for prevention of the probability of surgical adhesions, wherein the surfactant preparation comprises at least one phospholipid, pulmonary surfactant proteins SP-B and/or SP-C and/or modified derivatives thereof, and optionally excipients. The use of a surfactant preparation comprising at least one phospholipid, pulmonary surfactant proteins SP-B and/or SP-C and/or modified derivatives thereof and optionally excipients prevents that tissue surfaces—which normally slide with minimal friction—stick to each other. Thus, a surfactant preparation comprising at least one phospholipid, pulmonary surfactant proteins SP-B and/or SP-C and/or modified derivatives thereof and optionally excipients has an anti-adhesive activity. This modus operandi prevents formation of tissue bridges and restriction in movement, which inter alia prevents pain and hemorrhaging. Pain and hemorrhaging by itself can also lead to adhesions—a vicious circle which can be stopped by the use of a surfactant preparation of present invention.

It is a matter of course that a pharmaceutical composition comprising a surfactant preparation of at least one phospholipid, pulmonary surfactant proteins SP-B and/or SP-C and/or modified derivatives thereof and optionally excipients has the same surface activity and the same anti-adhesive activity in relation to surgical adhesions as the surfactant preparation itself.

According to this invention, the term “patient in need” refers to humans having the risk to develop surgical adhesions. As surgical adhesion may result from surgery as well as from other forms of skin injury or wounds, “patient in need” particularly refers to humans who are immediately prior to an operation or who are just operated or to humans who have been injured in their skin in such a way that the edges of the injured skin has to be held together by sutures. Particular mentioned is made to such humans who are immediately prior to a surgery of the abdomen or who are just operated at the abdomen. Also particularly mentioned are such humans who are immediately prior to a surgery of the thorax or who are just operated at the thorax. In particular patients during an intervention on the open thorax, and patients during an intervention on the open abdomen may be mentioned.

As an example, there is provided the use of a surfactant preparation of present invention in a patient having an intervention on the open thorax. Particular mention is made to the use of a surfactant preparation of present invention in a patient having an intervention on the heart such as a bypass operation or a heart valve operation.

Also exemplary patients in need are those patients to whom an intervention on the lungs is performed. Particular mention is made to lung transplantation or pneumonectomy. According to this invention, the probability of surgical adhesions in connection with the treatment of a patient to whom lungs are transplanted may be reduced by coating the lungs with a surfactant preparation of present invention prior to transplantation. This treatment is preferably carried out by using a powder formulation of a surfactant preparation and by topically administering the powder directly on the explanted organ prior to its implantation. The thoracic cavity of the patient to whom lungs are transplanted is also powdered with the surfactant preparation prior to transplantation.

Other exemplary patients in need are those patients having abdominal surgery such as—for example—a surgery on the bowel or colon.

Other exemplary patients in need are those who await a tendon surgery, whereby the adhesion of the tendon to the tendon sheath has to be avoided.

Other exemplary patients in need are those who await a facelift where deformity of skin tissues and underlying tissues has to be avoided.

According to the invention, the patient in need is preferably a patient who has not yet developed any surgical adhesion.

“Surfactant preparation” is understood according to the invention as meaning the numerous known compositions comprising phospholipids, pulmonary surfactant proteins and their modifications which compositions have the function of natural surfactant.

Natural surfactant has surface-active properties; it reduces, for example, the surface tension in the alveoli. A simple and rapid in vitro test with which the surface activity of surfactant can be determined is, for example, the so-called Wilhelmy balance [Goerke, J. Biochim. Biophys. Acta, 344: 241-261 (1974), King R. J. and Clements J. A., Am. J. Physicol. 223: 715-726 (1972)]. This method gives information on the pulmonary surfactant quality, measured as the action of a pulmonary surfactant of achieving a surface tension of almost zero mN/m. Another measuring device for determining the surface activity of surfactant is the pulsating bubble surfactometer [Possmayer F., Yu S. and Weber M., Prog. Resp. Res., Ed. v. Wichert, Vol. 18:112-120 (1984)].

Preferred compositions are those which, for example, have activity in the tests described above. Particularly preferred compositions are those which exhibit increased activity in such a test in comparison with natural, in particular human surfactant.

Preferred “phospholipids” according to the invention are dipalmitoylphosphatidylcholine (DPPC), palmitoyloleylphosphatidylglycerol (POPG) and/or phosphatidylglycerol (PG). Particularly preferably, the phospholipids are mixtures of various phospholipids, in particular mixtures of dipalmitoyl-phosphatidylcholine (DPPC) and palmitoyloleylphosphatidylglycerol (POPG), preferably in the ratio from 7 to 3 to 3 to 7.

Suitable “pulmonary surfactant proteins” are both the proteins obtained from natural sources, such as pulmonary lavage or extraction from amniotic fluid, and the proteins prepared by genetic engineering (recombinantly) or chemical synthesis. According to the invention, in particular the pulmonary surfactant proteins designated by SP-B (Surfactant Protein-B) and SP-C (Surfactant Protein-C) and their modified derivatives are of interest. The amino acid sequences of these pulmonary surfactant proteins, their isolation or recombinant preparation by genetic engineering are known (e.g. from WO 86/03408, EP 0251449, WO 89/04326, WO 87/06943, WO 88/03170, WO 91/00871, EP 0368823 and EP 0348967). Modified derivatives of the pulmonary surfactant proteins designated by SP-C, which differ from human SP-C by the replacement of a few amino acids, are described, for example, in WO 91/18015 and WO 95/32992. Particularly to be emphasized in this connection are the recombinant SP-C derivatives which are disclosed in WO 95/32992, in particular those which differ from human SP-C in positions 4 and 5 by the replacement of cysteine by phenylalanine and in position 32 by the replacement of methionine by isoleucine [designated herein as rSP-C (FF/I) or lusupultide (INN)]. “Modified derivatives” of pulmonary surfactant proteins are also understood as meaning those proteins which have a completely originally designed amino acid sequence with respect to their pulmonary surfactant properties, such as are described in EP 0593094 and WO 92/22315. Preferably, the polypeptide KL4 (INN: sinapultide) may be mentioned in this connection. The name pulmonary surfactant protein, according to the invention, also comprises mixtures of different pulmonary surfactant proteins.

According to present invention, surfactant preparations comprising one or more surfactant proteins are preferred.

It has been shown by in vitro experiments (either by using the Wilhelmy balance or the pulsating bubble surfactometer or the Maximum Bubble Pressure Tensiometer (MPT) [V. B. Fainerman and R. Miller, The maximum bubble pressure technique, monograph in “Drops and Bubbles in Interfacial Science”, in “Studies of Interface Science”, D. Möbius and R. Miller (Eds.), Vol. 6, Elsevier, Amsterdam, 1998, p. 279-326)]) that the addition of a surfactant protein, in particular rSP-C, to a surfactant preparation containing phospholipid reduces the surface tension and therefore enhances the efficacy of the composition with regard to an intensified sliding activity compared to a surfactant preparation solely containing phospholipids (see FIG. 1). The intensified modus operandi of the surfactant preparation containing phospholipid and surfactant protein rSP-C refers to the increased spreading activity due to the activity of rSP-C measured as a faster achievement of minimal surface tension in the Wilhelmy Balance or in the Pulsating Bubble Surfactometer or in the Maximum Bubble Pressure Tensiometer (MPT). Respectively, surfactant preparations of present invention allow movement between different tissues (e.g. in the thoracic cavity between the lungs and the pleura) at least equally good when compared to known phospholipid preparations. Particularly, surfactant preparations of present invention improve movement between adjacent tissues when compared to known phospholipid preparations. Thus, surfactant preparations of present invention and in particular those containing rSP-C are suitable for the treatment—prophylaxis and/or prevention—of surgical adhesions in patients in need thereof.

As further constituents or “excipients” which can be present in surfactant preparations, fatty acids such as palmitic acid may be mentioned. The surfactant preparations can also contain electrolytes such as calcium, magnesium and/or sodium salts (for example calcium chloride, sodium chloride and/or sodium hydrogencarbonate) in order to establish an advantageous viscosity. Preferred preparations according to the invention contain 80 to 95% by weight of phospholipids, 0.1 to 3.0% by weight of pulmonary surfactant proteins, 3 to 15% by weight of fatty acid, preferably palmitic acid, and 0 to 3% by weight of calcium chloride.

The surfactant preparations are prepared by processes known per se and familiar to the person skilled in the art, for example as described in WO 95/32992. According to the invention, the surfactant preparations can be lyophilized and spray-dried. Lyophilized preparations are disclosed, for example, in WO 97/35882, WO 91/00871 and DE 3229179. WO 97/26863 describes a process for the preparation of powdered pulmonary surfactant preparations by spray drying. Compositions of powdered surfactant preparations are exemplified in examples 1 to 6 of present invention.

According to this invention, administration of a surfactant preparation comprising at least one phospholipid, pulmonary surfactant proteins SP-B and/or SP-C and/or modified derivatives thereof and optionally excipients to a patient in need thereof has to be decided on a case-by-case basis in a way known per se and familiar to the person skilled in the art. Whether the surfactant preparation is administered as a powder, a suspension, a solution, or a paste or in the form of an atomization of a pulmonary surfactant solution or a pulmonary surfactant suspension or by atomization of pulmonary surfactant powder depends on the type and size of the intervention and thus on the type and size of the surgical adhesion.

In the case of a solution or suspension, the solution or suspension is prepared directly before use and bottled in a suitable device, preferably in a syringe, or in an ampoule, or in a squeeze bottle. The solution or suspension is administered topically directly onto the tissue concerned, i.e. each tissue or tissue layer in the cavity or skin injury or wound, in such a way that each tissue or tissue layer is coated by the suspension or solution. It is preferred that a suspension or solution comprising a surfactant preparation of present invention is administered by use of a syringe or a squeeze bottle.

Particular mention is made to the use of a lyophilized surfactant preparation comprising at least one phospholipid, pulmonary surfactant proteins SP-B and/or SP-C and/or modified derivatives thereof and optionally excipients, which lyophilized surfactant preparation is used as starting material for a suspension or solution of the surfactant preparation of present invention. According to this invention, the lyophilized surfactant preparation may be filled in a single-serving squeeze bottle in an amount beneficial for one use. Directly before use the lyophilized surfactant preparation is dissolved by addition of a suitable solvent. The dissolved surfactant preparation—an example of the herein referred pharmaceutical composition—is administered by use of the squeeze bottle. According to this invention, such a pharmaceutical composition may be used directly before and/or during closing a wound or incision or other form of skin injury to prevent the development of a surgical adhesion.

In the case of a pasty formulation of a surfactant preparation of present invention, the paste is administered topically onto the tissue concerned and due to its high viscosity distributed by an instrument, e.g. a spatula, in a way that the tissue or tissue layer is completely coated by the pasty formulation of the surfactant preparation.

It is particularly preferred that the surfactant preparation is formulated as a powder and administered topically to each tissue or tissue layer in the cavity or skin injury or wound in such a way that each tissue or tissue layer is coated by the powdered surfactant preparation. It is preferred that a powder comprising a surfactant preparation of present invention is administered by use of a device known in the art such as a squeeze bottle or a powder spray or a sifter-top package or a sifter-top container.

The application of a powder is especially preferred in patients having interventions on the open thorax or in patients having abdominal surgery. It has been shown that in cases of adhesions in large body areas a surfactant preparation formulated as a solution or suspension or paste is not suitable. A solution or suspension of a surfactant preparation of present invention may probably not adhere and may flow away from the injured area. It has been shown that the use of a paste may not be appropriate because of its high viscosity and hence the resulting need to coat the paste onto a large body area (tissue or tissue layer) by use of an instrument, e.g. a spatula, which is time-consuming and which may lead to additional problems such as infections in the injured body area or non-uniform spreading of the surfactant preparation. The use of a powdered surfactant preparation by use of a squeeze bottle or a powder spray or a sifter-top container can be done without directly contacting the injured body area thus reducing the risk of infection. The administration of surfactant preparation of present invention as a powdered formulation may result in an optimal spreading of the surfactant preparation over the tissues or tissue layers even in case of a large body area. It is also of advantage that the powdered surfactant preparation applied to an injured body area may be seen because of its white or yellow color helping the physician to uniformly apply the surfactant preparation.

As a result of the topical administration of a surfactant preparation of present invention, sliding of adjacent tissues or tissue layers is enhanced. Thus, present invention relates to a method for treating adhesions—preventing the probability of adhesions—in a patient in need thereof, the method comprises the step of topically administering a surfactant preparation of present invention to the patient in need thereof.

Preferably, surfactant preparations according to the invention are dissolved or suspended in a suitable solvent or resuspension medium, in particular if the preparations are present in lyophilized or spray-dried form. Preferably, the suitable resuspension medium is a physiological saline solution. It has proven advantageous to administer suspensions or solutions of the surfactant preparations which contain 25 to 100 mg of phospholipids per ml of suspension. Preferably, the surfactant preparations are administered per application in such an amount that the amount of phospholipids is between 12.5 and 200 mg per kilogram of body weight. It is preferred that the surfactant preparation of present invention contains 0.1 to 2.0 mg of rSP-C (FF/I) per ml of solvent. Particular mention may be made of a surfactant preparation containing 0.1 to 1.5 mg of rSP-C (FF/I) per ml of solvent.

In a method for treating adhesions in a patient in need thereof, the surfactant preparation of present invention is administered at least one time. It is preferred that the administration of a surfactant preparation of present invention for prophylaxis of adhesions and/or preventing the probability of adhesions is carried out once.

A further subject of present invention is a “commercial product”. According to present invention, the secondary packaging, the primary packaging comprising the pharmaceutical preparation and the patient pack of the commercial product correspond to what the person skilled in the art would regard as standard commercial product for pharmaceutical preparations of this type.

A suitable “primary packaging” depends on the formulation of the surfactant preparation but is principally known per se and familiar to the person skilled in the art. For example, a solution or suspension may be bottled in a syringe or a squeeze bottle or an ampoule, whereas a paste may be bottled in a bottle or a glass or a container and a powder formulation of the surfactant preparation may be bottled in a squeeze bottle or a powder spray bottle or a sifter-top container or a sifter-top package.

A suitable “secondary packaging” which may be mentioned by way of example is a folding box. Further packaging may also be such which are used to apply pastes.

### FIGURES

FIG. 1: The surface properties of surfactant preparations were studied by measuring the surface pressure at 1 second at 37° C. by Maximum Bubble Pressure Tensiometer (MPT) [V. B. Fainerman and R. Miller, The maximum bubble pressure technique, monograph in “Drops and Bubbles in Interfacial Science”, in “Studies of Interface Science”, D. Möbius and R. Miller (Eds.), Vol. 6, Elsevier, Amsterdam, 1998, p. 279-326)]. Measurements were performed with surfactant concentrations of 25 mg PL (Phospholipid)/ml. Surfactant preparation used are: (1) a phospholipid (PL) matrix; (2) Venticute® (INN: LUSUPULTIDE) (ALTANA Pharma AG), a synthetic surfactant containing rSP-C (FF/I); (3) EXOSURF® (INN: COLFOSCERIL PALMITATE) (Glaxo SmithKline), a synthetic phospholipid containing excipients; (4) SURVANTA® (INN: BERACTANT) (Abbott GmbH, Wiesbaden), extract of bovine lungs; (5) ALVEOFACT® (INN: BOVACTANT) (Boehringer Ingelheim), extract of bovine lungs; (6) INFASURF® (INN: CALFACTANT) (Forest Pharmaceuticals), a surfactant extracted from calf lungs; and (6) BLES® (BLES Biochemical Inc.), a bovine lipid extract surfactant.

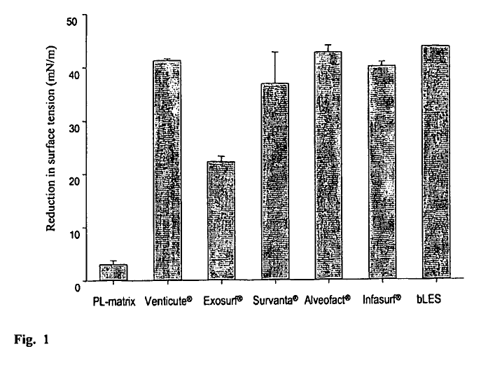

### EXAMPLES

### A.) Production of Powdered Surfactant Preparations

Powdered surfactant preparations are produced by the process described in WO 97/26863:

### Example 1

7.0 g of 1,2-dipalmitoyl-3-sn-phosphatidylcholine, 2.5 g of 1-palmitoyl-2-oleoyl-3-sn-phosphatidyl-glycerol sodium, 205 mg of calcium chloride dihydrate and 250 mg of palmitic acid are dissolved in 300 ml of ethanol/water (85:15) with warming to 60° C., cooled to room temperature and mixed with 350 ml of a solution of rSP-C (FF/I) in chloroform/methanol 9:1 (c=429 mg/1). The resulting solution is spray-dried in a Büchi B 191 laboratory spray dryer. Spray conditions: drying gas air, inlet temperature 90° C., outlet temperature 52-54° C. A relatively loose powder is obtained.

### Example 2

A solution of the surfactant obtained from bovine lungs (obtained by extraction and purification steps such as described, for example, in EP 406732) in chloroform/methanol is spray-dried under the following conditions: Büchi B 191 laboratory spray dryer, drying gas nitrogen, inlet temperature 80° C., outlet temperature 50-52° C. A fine, yellowish powder is obtained.

### Example 3

10.95 g of 1,2-dipalmitoyl-3-sn-phosphatidylcholine, 4.6 g of 1-palmitoyl-2-oleoyl-3-sn-phosphatidyl-glycerol ammonium, 418 mg of calcium chloride dihydrate and 750 mg of palmitic acid are dissolved in 330 ml of 2-propanol/water (85:15) at 50° C. and, after cooling to 30° C., mixed with 620 ml of a solution of rSP-C (FF/I) in isopropanol/water (95:5, c=484 mg/l). The resulting solution is spray-dried in a Büchi B 191 laboratory spray dryer. Spray conditions: drying gas nitrogen, inlet temperature 100° C., outlet temperature 58-60° C. A colorless powder is obtained.

### Example 4

3.74 g (5.1 mmol) of 1,2-dipalmitoyl-3-sn-phosphatidylcholine, 2.81 g (3.7 mmol) of 1-palmitoyl-2-oleoyl-3-sn-phosphatidylcholine, 2.90 g (3.9 mmol) of 1,2-dipalmitoylphosphatidyl-3-sn-phosphatidyl-glycerol sodium, 234 mg of palmitic acid and 279 mg (1.9 mmol) of calcium chloride dihydrate are dissolved in 160 ml of 2-propanol/water (85:15) at 50° C. and, after cooling to 30° C., mixed with 566 ml of a solution of rSP-C (FF/I) in isopropanol/water (92:8, c=330 mg/l) at 30° C. The resulting solution is spray-dried in a Büchi B 191 laboratory spray dryer. Spray conditions: drying gas nitrogen, inlet temperature 90° C., outlet temperature 58-60° C. A colorless powder is obtained.

### Example 5

0.5 g of KL4 (INN: sinapultide), 7.125 g of 1,2-dipalmitoyl-3-sn-phosphatidylcholine and 2.43 g of 1-palmitoyl-2-oleoyl-3-sn-phosphatidylglycerol ammonium are dissolved in 500 ml of chloroform/methanol 1:1 with warming to 45° C. and then spray-dried in a Büchi B 191 laboratory spray dryer. Spray conditions: drying gas nitrogen, inlet temperature 85° C., outlet temperature 55° C. A colorless powder is obtained.

### Example 6

A solution of phospholipids, palmitic acid and calcium chloride dihydrate obtainable according to Example 1, 3 or 4 is spray-dried—without addition of a solution of rSP-C (FF/I)— corresponding to the conditions according to Example 1, 3 or 4. A powder is obtained.

### B) Production of a Commercial Product

### Example 7

0.1 to 10 g of the powder obtained according to Example 1 is dispensed into a bottle of volume 100 to 250 ml and the bottle is sealed. The bottle is packed in a suitable folding box together with a package insert.

### Example 8

0.1 to 10 g of the powder obtained according to Example 1 is dispensed into a squeeze bottle of volume 100 ml and the squeeze bottle is sealed. The squeeze bottle is packed in a suitable folding box together with a package insert.

### Example 9

0.1 to 10 g of the powder obtained according to Example 1 is dispensed into a sifter-top container of volume 100 ml and the sifter-top container is sealed. The sifter-top container is packed in a suitable folding box together with a package insert.

### C) Use of a Powdered Surfactant Preparation

### Example 10

The squeeze bottle of Example 8 is unpacked, unsealed and the powdered surfactant preparation is applied directly onto the injured body area by pushing the squeeze bottle and thereby ejecting the powdered surfactant preparation out of the squeeze bottle.

### Example 11

The sifter-top container of Example 9 is unpacked, unsealed and the powdered surfactant preparation is applied directly onto the injured body area by shaking the sifter-top container and thereby ejecting the powdered surfactant preparation out of the sifter-top container directly onto the tissue concerned.

1-6. (canceled)

7. A method for treating surgical adhesions in a patient in need thereof comprising the step of:
administering a surfactant preparation comprising at least one phospholipid, surfactant protein and optionally excipients to the patient in need thereof.

8. The method according to claim 7, wherein the surfactant preparation is administered topically.

9. A pharmaceutical composition comprising a surfactant preparation suited for a method of claim 7, wherein the surfactant preparation comprises at least one phospholipid, pulmonary surfactant proteins SP-B and/or SP-C and/or modified derivatives thereof, and optionally excipients.

10. A commercial product comprising:
a customary secondary packaging,
a primary packaging comprising a pharmaceutical preparation of at least one phospholipid and surfactant protein SP-B and/or SP-C and, optionally,
a package insert,
the pharmaceutical preparation being suitable for treatment of surgical adhesions in patients in need thereof.

11. A pharmaceutical composition comprising a surfactant preparation suited for the method of claim 8, wherein the surfactant preparation comprises at least one phospholipid, pulmonary surfactant proteins SP-B and/or SP-C and/or modified derivatives thereof, and optionally excipients.

12. The method according to claim 7, wherein the surfactant protein is recombinantly prepared pulmonary surfactant protein.

13. The method according to claim 7, wherein the surfactant protein is a modified derivative of a pulmonary surfactant protein.

14. The method according to claim 12, wherein the pulmonary surfactant protein is rSP-C (FF/I).

15. The method according to claim 7, wherein the surfactant preparation is in the form of a powder.

16. The method according to claim 15, wherein the powder is obtainable by spray-drying.
